(19) United States
(12) Patent Application Publication
(10) Pub. No.: US 2023/0230683 A1
(43) Pub. Date: Jul. 20, 2023

(54) PATIENT TRANSFER IN VIRTUAL MEDICINE

(71) Applicant: BANTY, INC., SCAROROUGH (CA)

(72) Inventors: Mariya BOYKO, OAKVILLE (CA); Scott William WILSON, BURLINGTON (CA); Richard Henry TYTUS, BURLINGTON (CA); Marc TYTUS, HAMILTON (CA); Yasith Prabuddhaka Peduru Hewage, Bataganvilla (LK)

(21) Appl. No.: 18/082,667

(22) Filed: Dec. 16, 2022

(30) Foreign Application Priority Data
Dec. 20, 2021 (CA) 3143139

(51) Int. Cl. G16H 40/20; G16H 40/67

(52) U.S. Cl. CPC G16H 40/20; G16H 40/67

(57) ABSTRACT

A join request from a patient computing system identifies a patient, who is assigned to a waiting pool exposing each patient therein to a provider admitting computing system. An admission selection of the patient is received from the provider admitting computing system, and an audiovisual admissions teleconference between the patient computing system and the provider admitting computing system is initiated. A practitioner selection assignment from the provider admitting computing system assigns the patient to a provider practitioner, and in response, the patient is assigned to a provider practitioner pool associated with a provider practitioner computing system. The provider practitioner pool exposes each patient therein to the provider practitioner computing system. The audiovisual admissions teleconference is terminated. A first consultation selection of the patient from the first provider practitioner computing system initiates a first audiovisual consultation teleconference between the patient computing system and the provider practitioner computing system.

### TECHNICAL FIELD

The present disclosure relates to telemedicine.

### BACKGROUND

Telemedicine (the ability to provide medical services at a distance using computerized audiovisual connections) has been in use for several years. Telemedicine has risen in popularity as a result of the SARS-COVID-19 pandemic which emerged in early 2020. Doctors and other medical professionals found themselves needing to adapt to the new workflow and to incorporate virtual medicine into their daily practice.

One problem with telemedicine is the lack of administrative assistance when taking video calls with patients throughout the day. The doctor or other medical professional is currently responsible for taking care of administrative tasks on the call, whereas in an in-person physical clinic, they are able to delegate those tasks to receptionists and nurses, and to help more patients as a result. Doctors may become burnt out and unproductive because of the inability to delegate administrative tasks to their staff when undertaking virtual consultations with their patients.

There are technical difficulties in integrating this important administrative support into conventional videoconferencing platforms, including network limitations. In addition, patients may find virtual clinic visits confusing because of lengthy URLs. Virtual clinic visits may be especially challenging for older patients who are not accustomed to using telemedicine, and are unfamiliar with digital devices. Thus, there are acute problems associated with conforming telemedicine to the workflow and experience that patients and medical practitioners have come to expect. These problems are inherent to conventional telemedicine, and are by their nature a form of computer problem requiring the application of technology to generate a satisfactory resolution.

### SUMMARY

The present disclosure describes systems, methods and computer program products that enable health care providers offering telemedicine services to use a workflow that replicates the real-life clinic experience, and to provide patients with relevant information.

In one aspect a method for administering telemedicine consultations is provided. The method comprises receiving, from a patient computing system, a join request for a patient consultation, wherein the join request identifies a patient associated with the patient computing system. Responsive to the join request, the method assigns the patient to a waiting pool, wherein the waiting pool exposes each patient in the waiting pool to a provider admitting computing system. The method receives, from the provider admitting computing system, an admission selection of the patient assigned to the waiting pool, and, responsive to the admission selection, initiates an audiovisual admissions teleconference between the patient computing system and the provider admitting computing system. The method further receives, from the provider admitting computing system, a first practitioner selection assignment assigning the patient to a first provider practitioner, and, responsive to the first practitioner selection assignment, assigns the patient to a first provider practitioner pool associated with a first provider practitioner computing system associated with a first provider practitioner. The first provider practitioner pool exposes each patient in the first provider practitioner pool to the first provider practitioner computing system. The method terminates the audiovisual admissions teleconference, receives, from the first provider practitioner computing system, a first consultation selection of the patient in the first provider practitioner pool, and, responsive to the first consultation selection, initiates a first audiovisual consultation teleconference between the patient computing system and the first provider practitioner computing system.

In one embodiment, assigning the patient to a waiting pool comprises assigning a patient object representing the patient to a waiting queue, assigning the patient to the first provider practitioner pool comprises assigning the patient object representing the patient to a first provider practitioner queue, and the waiting queue and the first provider practitioner queue are distinct from one another.

In another embodiment, assigning the patient to a waiting pool comprises assigning a patient object representing the patient to a waiting queue, assigning the patient to a first provider practitioner pool comprises assigning the patient object representing the patient to a common practitioner provider queue and assigning a first provider practitioner tag to the patient object. The first provider practitioner tag associates the patient object with the first provider practitioner computer system, and the waiting queue and the common provider practitioner queue are distinct from one another.

In yet another embodiment, assigning the patient to a waiting pool comprises assigning a patient object representing the patient to a single common queue and assigning an admitting tag to the patient object, and assigning the patient to the first provider practitioner pool comprises maintaining the patient object in the single common queue and assigning a first provider practitioner tag to the patient object. The first provider practitioner tag associates the patient object with the first provider practitioner computer system.

The method may further comprise receiving, from the first provider practitioner computing system, a second practitioner selection assignment assigning the patient to a second provider practitioner. Responsive to the second practitioner selection assignment, the method assigns the patient to a second provider practitioner pool associated with a second provider practitioner computing system. The second provider practitioner pool exposes each patient in the second provider practitioner pool to the second provider practitioner computing system. The method terminates the first audiovisual consultation teleconference, receives, from the second provider practitioner computing system, a second consultation selection of the patient in the second provider practitioner pool, and, responsive to the second consultation selection, initiates a second audiovisual consultation teleconference between the patient computing system and the second provider practitioner computing system.

In one embodiment, the first provider practitioner pool exposes each patient in the first provider practitioner pool to the second provider practitioner computing system and the second provider practitioner pool exposes each patient in the second provider practitioner pool to the first provider practitioner computing system.

In one embodiment, wherein the waiting pool further exposes each patient in the waiting pool to the first provider practitioner computing system.

The method may further comprise, responsive to the admission selection, assigning the patient to an admissions pool in association with initiating the audiovisual admissions teleconference between the patient computing system and the provider admitting computing system.

In another aspect, a method of providing patient-centered medical information, comprising. While a patient waits to enter a virtual consultation in a telemedicine platform, the method receives medical data from a patient computing system associated with the patient, and uses the medical data to select at least one targeted media item. The targeted media item(s) are selected for relevance to the patient based on the medical data. The method presents to the patient, in a user interface of the patient computing system, an opportunity to make a selection of one of (a) the targeted media item(s); and (b) at least one distractor media item of lesser relevance to the patient, relative to the targeted media item(s). The method receives the selection from the patient computing system, and responsive to the selection, presents, in the user interface of the patient computing system, the selected one of the targeted media item(s) and the distractor media item(s).

In one embodiment, the method may further comprise using the medical data to select the distractor media item(s) to be of lesser relevance to the patient, relative to the at least one targeted media item. In another embodiment, the at least one distractor media item(s) may be selected randomly.

In one embodiment, using the medical data to select at least one targeted media item comprises applying a decision tree to the medical data.

In one embodiment, using the medical data to select at least one targeted media item comprises applying a trained machine learning engine to the medical data.

In other aspects, telemedicine computing systems and computer program products for implementing the above methods are provided.

### BRIEF DESCRIPTION OF THE DRAWINGS

These and other features will become more apparent from the following description in which reference is made to the appended drawings wherein:

FIG. 1 shows an overview schematic of an illustrative telemedicine computing system according to the present disclosure;

FIGS. 1A to 1C show queue-based embodiments of waiting pools and provider practitioner pools;

FIG. 2 is a class diagram showing certain models in an embodiment of an object-oriented queue-based implementation according to the present disclosure;

FIG. 3 is an illustrative class diagram showing models relating to one illustrative queue-based implementation of an audiovisual consultation teleconference;

FIG. 4 is a flow chart showing an illustrative method for administering telemedicine consultations;

FIG. 5 illustrates a system for providing patient-centered medical information while a patient waits to enter a virtual consultation in a telemedicine platform;

FIG. 6 is a flow chart showing an illustrative method of providing patient-centered medical information while a patient waits to enter a virtual consultation in a telemedicine platform;

FIG. 7 is a block diagram showing an illustrative computer system in respect of which aspects of the present disclosure may be implemented; and

FIG. 8 is a block diagram showing an illustrative networked wireless telecommunication computing device in respect of which aspects of the present disclosure may be implemented.

### DETAILED DESCRIPTION

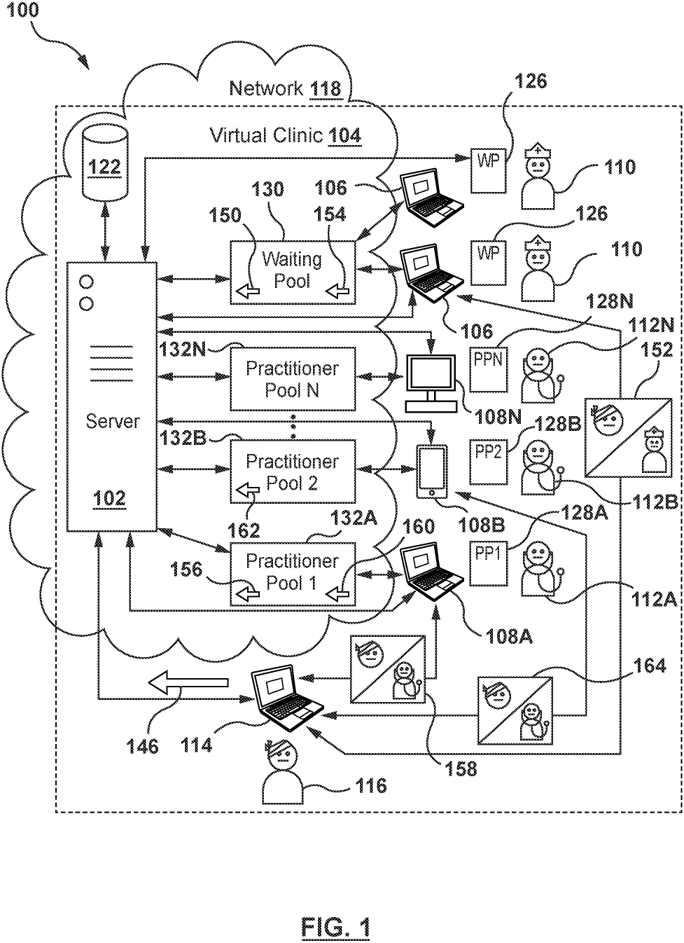

Reference is now made to FIG. 1, which shows an overview schematic of an illustrative telemedicine computing system, indicated generally by reference 100. The telemedicine computing system 100 comprises a telemedicine server system 102. Although shown as a single computer for illustrative services, the telemedicine server system 102 may comprise a plurality of individual computers, which may be co-located in a single data center, or distributed among a plurality of geographic locations. No architectural limitation is implied.

The telemedicine server system 102 is communicatively coupled to a plurality of other computing devices to form a virtual clinic 104. The devices to which the telemedicine server system 102 include a plurality of provider admitting computing systems 106 associated with a provider, and a plurality of provider practitioner computing systems 108A, 108B...108N associated with the provider. While a plurality of provider admitting computing systems 106 and a plurality of provider practitioner computing systems 108A, 108B...108N are shown for purposes of illustration, in some embodiments there may be only a single provider admitting computing system and/or only a single provider practitioner computing system within a virtual clinic. The provider admitting computing systems 106 and the provider practitioner computing systems 108A, 108B...108N may be any type of computing device, including without limitation a desktop computer, laptop computer, tablet computer, smartphone, or other device.

The term “provider”, as used herein, refers to an entity that provides medical services for which at least some component of those services may be delivered remotely by telemedicine. For example, a surgical practice may administer pre-surgical and post-surgical consultation remotely by telemedicine even if surgery is performed in person. A provider may be, for example, a medical practice incorporating one or more general practitioners, or one or more specialists, and includes a psychiatrist or psychologist practice, chiropodist practice, a podiatrist practice, an occupational therapy practice, a physiotherapy practice, a chiropractic practice, a dental (including orthodontic) practice as well as a veterinary practice. A provider may have a physical clinic, such as a doctor’s office or surgical suite, or an office within a hospital, to which the virtual clinic 104 is an adjunct, or may in some circumstances practice exclusively through the virtual clinic.

The provider admitting computing systems 106 are used by provider staff members 110. The provider staff members 110 may be a medical professional, such as a nurse, or an allied professional such as a secretary or receptionist. There may be a plurality of provider staff members 110 as shown, or a single provider staff member. The provider practitioner computing systems 108A, 108B...108N are used by respective provider practitioners 112A, 112B... 112N who are medical professionals, e.g. a doctor, dentist, veterinarian, etc. The provider admitting computing systems 106 and the provider practitioner computing systems 108A, 108B...108N may all be located at a single site where in-person physical medical services are provided, or some of the provider admitting computing systems 106 and/or some of the provider practitioner computing systems 108 may be located at the physical site and others may be located remotely, e.g. some or all of the provider staff members 110 and/or provider practitioners 112A, 112B...112N may work remotely from the site where physical medical services are provided. In some embodiments there may be no such physical site (i.e. an exclusively virtual practice).

The telemedicine server system 102 is also communicatively coupled to at least one patient computing system 114. Although only a single patient computing system 114 is shown for simplicity of illustration, there may be a plurality of patient computing systems 114. Each of the patient computing systems 114 is used by a particular patient 116 of the provider. A patient computing system 114 may be any type of computing device, including without limitation a desktop computer, laptop computer, tablet computer, smartphone, or other device. The patient computing device 114 is typically a remote patient computing device, that is, the patient computing device is typically in a different physical location from the provider admitting computing systems 106 and the plurality of provider practitioner computing systems 108A, 108B...108N, and is in a different physical location than the physical site, if any, where in-person physical medical services are provided.

The telemedicine server system 102 is communicatively coupled to the provider admitting computing systems 106, the provider practitioner computing systems 108A, 108B...108N and the patient computing system(s) 114 via a network 118, such as the Internet. The provider admitting computing systems 106 and the provider practitioner computing systems 108A, 108B...108N may be coupled to one another, and optionally to one or more local servers, via an intranet that is in turn coupled to the network 118. The telemedicine server system 102 is also communicatively coupled to a datastore 122.

The telemedicine server system 102 may be hosted in a cloud computing environment, for example Amazon Web Services (AWS). Alternatively, dedicated hardware for the telemedicine server system 102 may be provided. In one embodiment, the telemedicine server system 102 implements two main applications: a frontend or client-side application, and a backend or server-side application. The frontend application consists of the user interface (UI) and components with UI logic and may be implemented, for example, as a web application using the React JavaScript library and HTML for execution in a web browser on the provider admitting computing systems 106, the provider practitioner computing systems 108 and the patient computing system(s) 114. The backend application contains the components of the Application Programming Interface (API) server, and may be implemented, for example, using the Node.js server environment and a MongoDB application. As shown, the telemedicine server system 102 is communicatively coupled to a datastore 122. In one embodiment, the datastore 122 comprises MongoDB Atlas cloud storage; other cloud storage or dedicated local storage may also be used. In a preferred embodiment, connection from the API server backend application executing on the telemedicine server system 102 to the datastore 122 is secured. The credentials and the SRV link are included in the environment files on the telemedicine server system 102 so that when hosted in a secured cloud computing environment, the credentials and SRV link are protected by the cloud security protocols (e.g. AWS). Environment files are accessed at runtime. Similarly, contents of the datastore 122 may be encrypted so as to be accessible only with valid credentials, and further security can be provided by restricting access to the datastore to only whitelisted locations; both features are available in MongoDB. Also preferably, the datastore 122 is periodically backed up.

In one embodiment, communication between the frontend application and the backend application is by way of a representational state transfer (REST or RESTful) API using Hypertext Transfer Protocol Secure (HTTPS) to provide security. In one embodiment, the OAuth 2.0 authentication standard is used to provide authentication for the REST API. This allows the frontend application to be issued an access token by the telemedicine server system 102 to enable access to the desired resources; a client token is provided by the server when the user is logged in. The frontend application (user) includes that token with every request and the token is validated each time the frontend application requests data from the API server backend application before data is sent. Additional steps to check the token validity when using the REST API include testing that the token contains valid content, correlating the token with the requesting user identifier to confirm that a request is coming from the original user to whom the token was issued, and setting tokens to expire after a predetermined time (which may be longer if a “Keep Me Logged In” type feature is enabled). After expiry, a new token may be provided. Thus, in preferred embodiments communicative coupling between the telemedicine server system 102 and the provider admitting computing systems 106, the provider practitioner computing systems 108 and the patient computing system(s) 114 is secured.

In one embodiment, the backend application may cooperate with a separate conferencing application to support audiovisual teleconferences. The conferencing application preferably requires authentication before an audiovisual teleconference begins. This may be handled by a conference token generated at the time of the audiovisual teleconference start, which is used to verify the users’ validity with the conferencing application. The conference token is secure since the application is served compiled to a single JavaScript bundle. In other embodiments, conferencing functionality may be incorporated within the backend application.

The telemedicine server system 102 is adapted to maintain a plurality of pools representing patient status, including at least one provider waiting pool 130 and at least one provider practitioner pool 132A, 132B...132N. In a typical embodiment there will be a single provider waiting pool 130 and one or more provider practitioner pools 132A, 132B...132N each associated with a respective provider practitioner 112A, 112B...112N. The provider waiting pool(s) 130 and provider practitioner pool(s) 132A, 132B...132N are configured to mimic, in a telemedicine context, a clinic’s waiting room(s) and examination room(s), respectively. In a physical clinic, a patient would typically enter the waiting room, and (often after a period of time, depending on whether there are other patients) check in with the receptionist, who can then assign the patient to an examination room to await consultation with the relevant practitioner. Similarly, in the telemedicine computing system 100, a patient can be assigned to a provider waiting pool 130 and, after checking in, be assigned to a provider practitioner pool 132A, 132B...132N. The telemedicine server system 102 is adapted to expose 126 each patient in the provider waiting pool 130 to the provider admitting computing systems 106, and to respectively expose 128A, 128B...128N each patient 116 in the provider practitioner pools 132A, 132B...132N to at least the respective one of the provider practitioner computing systems 108A. 108B ...108N associated with the respective provider practitioner 112A, 112B...112N with whom the respective provider practitioner pool 132A, 132B...132N is associated.

In one embodiment, all of the pools, that is, the provider waiting pool(s) 130 and the provider practitioner pool(s) 132A, 132B...132N are exposed to all of the provider admitting computing systems 106 and all of the provider practitioner computing systems 108. Thus, a first provider practitioner pool 132A exposes each patient in the first provider practitioner pool 132A to a second provider practitioner computing system 108B through to an N^{th} provider practitioner computing system 108N and a second provider practitioner pool 132B exposes each patient in the second provider practitioner pool 132B to the first provider practitioner computing system 108A through to the N^{th} provider practitioner computing system 108N. Similarly, the waiting pool 130 further exposes each patient in the waiting pool 130 to the first provider practitioner computing system 108A, the second provider practitioner computing system 108B through to an N^{th} provider practitioner computing system 108N. Additionally, the first provider practitioner pool 132A exposes each patient in the first provider practitioner pool 132A to the provider admitting computing systems 106 and the second provider practitioner pool 132B similarly exposes each patient in the second provider practitioner pool 132B to the provider admitting computing systems 106. Alternatively, restrictions may be imposed on the provider admitting computing systems and the provider practitioner computing systems to which particular ones of the provider waiting pool(s) and the provider practitioner pool(s) are exposed.

The provider waiting pool 130 and provider practitioner pools 132A, 132B...132N can be configured and administered in a number of ways. In one general embodiment, the provider waiting pool 130 and provider practitioner pools 132A, 132B...132N are set up using queues, using an object-oriented data structure in which the queue and the patient are each represented as an object.

In one particular embodiment, as shown in FIG. 1A, there is a single provider waiting queue 130A and one or more separate provider practitioner queues 134A, 134B... 134N, with each of the provider practitioner queues 134A, 134B... 134N being associated with a specific respective practitioner. Objects representing patients 116 (patient objects 116A) are assigned to the waiting queue 130A and one or more separate provider practitioner queues 134A, 134B ... 134N. In this embodiment, the provider waiting queue 130A is separate and distinct from each of the provider practitioner queues 134A, 134B...134N. In this embodiment, the queues are preferably unique data structures that resemble physical patient queues in a physical clinic. The patient 116 cannot determine the next queue that s/he is going to transfer to.

In another embodiment, as shown in FIG. 1B, there is a single provider waiting queue 130B and a single common provider practitioner queue 136. Within the single common provider practitioner queue 136, each object representing a patient 116 (patient object 116B) has a provider practitioner tag 140B that associates the patient 116 with a particular provider practitioner 112A, 112B... 112N. Since each provider practitioner 112A, 112B... 112N is associated with a specific respective provider practitioner computer system 108, the provider practitioner tag 140B will therefore associate each patient 116 with a particular provider practitioner computer system 108. In this embodiment, the single provider waiting queue 130B is separate and distinct from the single common provider practitioner queue 136.

In yet another embodiment, as shown in FIG. 1C, there is a single common queue 142. The objects representing patients 116 (patient objects 116C) in the single common queue 142 may be assigned an admitting tag 144C to denote that they are in the waiting pool, or may be assigned a provider practitioner tag 140C that associates the patient 116 with a particular provider practitioner 112A, 112B... 112N and hence with a particular provider practitioner computer system 108A, 108B ...108N. Although not shown, it is also contemplated that there may be multiple waiting queues (e.g. there may be multiple provider staff members 110 each having their own waiting queue). At the code level, shorter data structures are preferred for the queues as this can improve performance.

The embodiment shown in FIG. 1A is currently preferred; at the code level this structure facilitates grouping the patients assigned to a single provider practitioner 112A, 112B...112N, checking whether a provider practitioner 112A, 112B...112N is busy with an assigned patient, and showing available provider practitioners 112A, 112B...112N (e.g. show the list of provider practitioner queues 134A, 134B...134N as a list of provider practitioners 112A, 112B...112N).

In one embodiment, the Firebase platform is used to provide real time updated component functionality for the queue system, as well as for messaging and notification features. The Firebase platform uses HTTPS calls and secured websocket communication, to thereby further secure the communicative coupling between the telemedicine server system 102 and the provider admitting computing systems 106, the provider practitioner computing systems 108A, 108B...108N and the patient computing system(s) 114.

The queue details are stored within the database layer (e.g. MongoDB) of the backend application and the real time queue updates are stored in the Firebase Firestore. The Firestore can maintain websocket connections and deliver snapshots of datasets when data is updated, enabling the frontend application (e.g. React and HTML) to capture the real time patient updates. The queue data structure in Firebase Firestore can be updated when a patient computing system 114 submits a join request 146, is assigned to a queue, or begins or ends an audiovisual admissions teleconference 152 or audiovisual consultation teleconference 158, 164. These changes can be detected at the frontend application, which can notify the provider practitioners 112A, 112B...112N, for example via a user interface change. Where the provider waiting pool 130 and provider practitioner pools 132A, 132B...132N are set up using multiple queues, each queue preferably behaves individually, that is, one queue will preferably not depend on any other queue’s data. Once a patient 116 is assigned to a queue, there is no link to the patient object 116A, 116B with the previous queue to which the patient 116 was assigned.

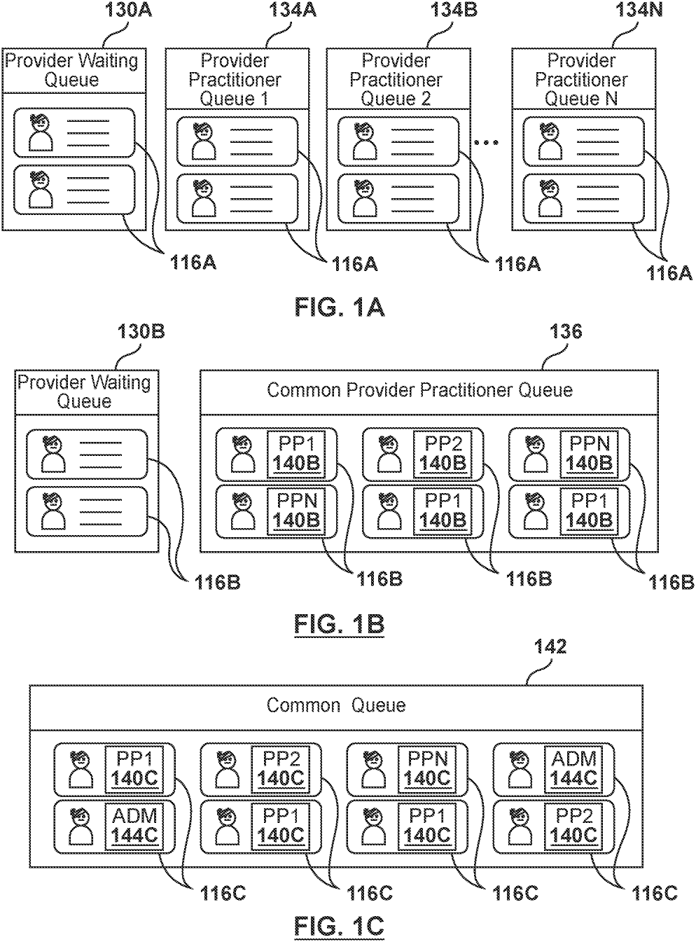

Reference is now made to FIG. 2, which is a class diagram 200 showing certain models in an embodiment of an object-oriented queue-based implementation. Provider entity 202 and provider practitioner entity 204 have database relations with the queue entity 206. A provider entity 202 may have multiple provider practitioners 204, and a provider practitioner entity 204 may have multiple queue entities 206. A queue entity may or may not be assigned to a provider practitioner entity 204.

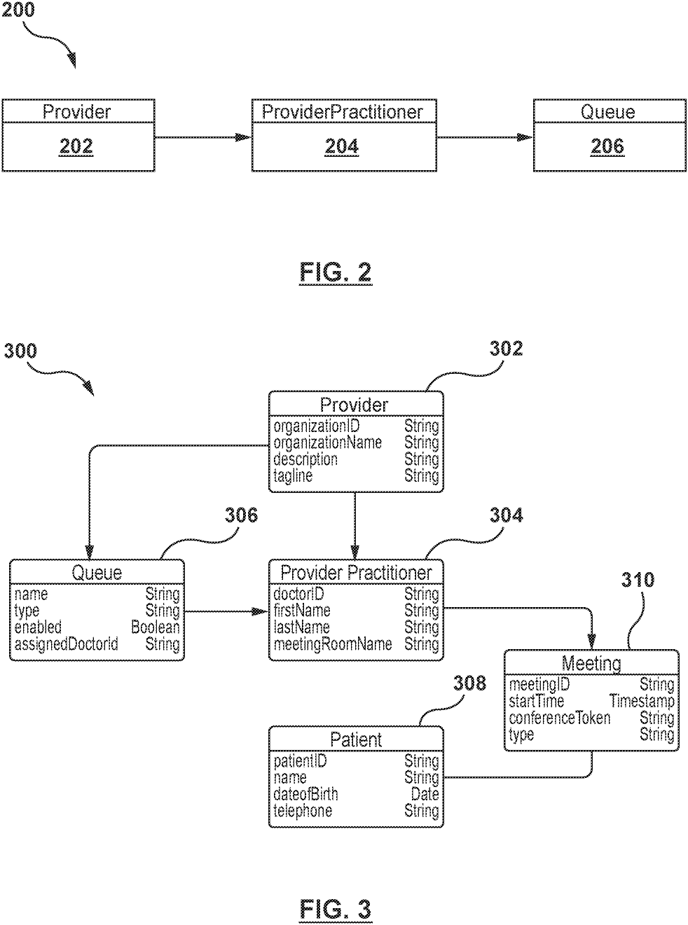

FIG. 3 is an illustrative class diagram 300 showing models relating to one illustrative queue-based implementation of an audiovisual consultation teleconference, including a provider entity 302, a provider practitioner entity 304, a queue entity 306, a patient entity 308 and a meeting entity 310.

Returning to FIG. 1, the telemedicine server system 102 receives, from the patient computing system 114, a join request 146 for a patient consultation. The join request 146 identifies the patient 116 associated with the patient computing system 114. In one embodiment, the patient 116 may be provided, ahead of a scheduled consultation, with a patient invite Uniform Resource Locator (URL) containing the domain and the virtual meeting name (e.g. https://medical.banty.me/kevins-clinic-384344407714422). This domain is the location where the frontend application is hosted and the virtual meeting name is the meeting reference associated with the provider who generated the patient invite URL. In one embodiment, when the patient visits the patient invite URL, the frontend application includes functionality to check audiovisual connections to ensure that the audiovisual input/output methods used by the patient computing system 114 are compatible with those supported by the telemedicine server system 102 (or separate conferencing system). The input/output methods are detected through the browser’s API services. The patient 116 may be provided with live feedback for the input/output methods. Optionally, this step may be performed at a later stage. The frontend application will preferably enable the patient 116 to use the patient computing system 114 to capture a digital photograph of the patient 116 (e.g. using a webcam or integrated camera). The photograph can be previewed to a provider staff member 110 and/or a provider practitioner 112A, 112B...112N (e.g. displayed alongside the name of the patient in an onscreen representation of a provider waiting pool 130 and/or provider practitioner pool 132A, 132B...132N). Preferably, the patient invite URL is only valid for a particular patient consultation, that is, unique to a particular patient/provider/practitioner/time slot.

In response to the join request 146, the telemedicine server system 102 assigns the patient 116 to the provider waiting pool 130. In the embodiments shown in in FIGS. 1A and 1B, assigning the patient to the provider waiting pool 130 comprises assigning the patient 116 to a waiting queue 130A, 130B (via patient object 116A, 116B) and in the embodiment shown in FIG. 1C, assigning the patient 116 to the waiting pool comprises assigning the patient to a single common queue 140 (via patient object 116C) and assigning an admitting tag 144C to the patient 116. The patient 116 may wait in the provider waiting pool 130 for a period of time, depending on whether there are other patients already waiting in the provider waiting pool 130 and the availability of provider staff members 110. In one embodiment, as described further below, a method of providing patient-centered medical information may be implemented for the patient 116 while the patient 116 is in the provider waiting pool 130.

When a provider staff member 110 is ready to admit the patient 116, that provider staff member 110 can make an admission selection 150 of the patient 116 assigned to the provider waiting pool 130. For example, the provider staff member 110 may select the patient 116 from a list displayed on a screen of the provider admitting computing systems 106.

The telemedicine server system 102 receives the admission selection 150 from the provider admitting computing system 106. In response to the admission selection 150, the telemedicine server system 102 initiates an audiovisual admissions teleconference 152 between the patient computing system 114 and the provider admitting computing system 106. Optionally, checking the audiovisual connections to ensure that the audiovisual input/output methods used by the patient computing system 114 are compatible with those supported by the telemedicine server system 102 can be carried out at this stage, e.g. immediately before initiating the audiovisual admissions teleconference 152. The audiovisual admissions teleconference 152 may connect the patient computing system 114 and the provider admitting computing system 106 through the telemedicine server system 102 (e.g. conference application servers may form part of the telemedicine server system 102), or the telemedicine server system 102 may mediate the audiovisual admissions teleconference 152 through an conferencing system or an external conferencing provider. Browser APIs may be used to detect the input/output methods in the provider admitting computing system 106. In one embodiment, the meeting contains a conferencing application (e.g. meet.jit.si) embedded into the main application, and when two users are in a meeting, the conferencing application maintains the connectivity and the audio/video streams of data.

After the audiovisual admissions teleconference 152 is initiated, the provider staff member 110 and the patient 116 see each other’s audiovisual input feeds. The audiovisual admissions teleconference 152 enables the provider staff member 110, such as a receptionist or nurse, to communicate with the patient 116. The provider staff member 110 may communicate with the patient 116 to obtain some basic details about the patient’s medical issue, and may also carry out administrative tasks, such as obtaining information about health insurance, government healthcare coverage, or even payment information (e.g. in the case of concierge medical services or other forms of private care, including veterinary care). Other matters may also be discussed. The audiovisual admissions teleconference 152 provides a digital simulacrum, within a digital medical clinic, of the scenario in a physical medical clinic where a patient interacts with a receptionist, nurse or other staff member before being transferred to a physician or other practitioner.

Optionally, the provider staff member 110 can invite one of the provider practitioners 112A, 112B...112N (or another user) to join the audiovisual admissions teleconference 152. This could be achieved, for example, by sharing the meeting name and the conference token with the relevant provider practitioner computing system 108A, 108B...108N within the application, with the application notifying the invited provider practitioner 112A, 112B... 112N, who can be provided with the option to accept or decline the invitation. In this embodiment, a notification may be sent to the provider practitioner 112A, 112B...112N (or other user), which would typically occur outside of the conferencing application (or conferencing functionality within the backend application). For example, the React application may deliver the notification, and will integrate the provider practitioner 112A, 112B...112N (or other user) into the audiovisual admissions teleconference 152 only if the invitation is accepted. The conferencing application (or conferencing functionality within the backend application) is configured such that so long as there are at least two participants in the audiovisual admissions teleconference 152, any additional participants can leave the audiovisual admissions teleconference 152 without disrupting it. For example, a provider staff member 110 can leave an audiovisual admissions teleconference 152 after introducing the provider practitioner 112 to the patient 116. This will simply remove the provider staff member 110 reference from the audiovisual admissions teleconference 152 and stop the media transport with the conferencing service. The conferencing application will continue without disrupting the audiovisual admissions teleconference 152 between the others.

More typically, however, once the provider staff member 110 has completed his/her interaction with the patient 116, the provider staff member 110 can provide to the telemedicine server system 102 a first practitioner selection assignment 154 assigning the patient to a particular provider practitioner 112A, 112B... 112N, in this case a first provider practitioner 112A. This can be done using any suitable user interface. The provider staff member 110 can include a note with the first practitioner selection assignment 154; this note would not be visible to the patient 116.

The telemedicine server system 102 receives the first practitioner selection assignment 154 from the provider admitting computing system 106 and, responsive to the first practitioner selection assignment 154, assigns the patient 116 to a first provider practitioner pool 132A associated with the first provider practitioner computing system 108A. For example, where there are multiple provider practitioners 112A, 112B... 112N, the patient 116 can be assigned to a provider practitioner pool 132A, 132B...132N associated with the provider practitioner computing system 108A, 108B...108N that is associated with a selected provider practitioner 112A, 112B... 112N, or where there is a single provider practitioner and a single provider practitioner pool, assign the patient to that single provider practitioner pool. In the embodiment shown in FIG. 1A, where there is a single provider waiting queue 130A and one or more separate provider practitioner queues 134A, 134B...134N each for a respective provider practitioner 112A, 112B... 112N, assigning the patient object 116 to the first provider practitioner pool 132A comprises assigning the patient object 116A to a first provider practitioner queue 134A. In the embodiment shown in FIG. 1B, where there is a single provider waiting queue 130B and a single common provider practitioner queue 136, assigning the patient 116 to the first provider practitioner pool 132B comprises assigning the patient object 116B to the common practitioner provider queue 136 and assigning a first provider practitioner tag 140B to the patient object 116B. The first provider practitioner tag 140B associates the patient object 116B, and hence the corresponding patient 116, with the first provider practitioner computer system 108. In an object-oriented implementation using multiple queues (FIGS. 1A and 1B), this will move the patient object 116A, 116B within the queue object structure at the database level. In the embodiment shown in FIG. 1C, where there is a single common queue 142, assigning the patient 116 to the first provider practitioner pool 132A comprises maintaining the patient object 116C in the single common queue 142 and assigning a first provider practitioner tag 140C to the patient object 116. The first provider practitioner tag 140C associates the patient object 116C, and hence the corresponding patient 116, with the first provider practitioner computer system 108A. As noted above, the first provider practitioner pool 132A exposes each patient in the first provider practitioner pool 132A to the first provider practitioner computing system 108A.

The telemedicine server system 102 will terminate the audiovisual admissions teleconference 152 based on suitable input. In one embodiment, the telemedicine server system 102 will terminate the audiovisual admissions teleconference 152 automatically in response to receiving the first practitioner selection assignment 154. In another embodiment, the telemedicine server system 102 will terminate the audiovisual admissions teleconference 152 in response to a command from the provider admitting computing system 106, either before or after the first practitioner selection assignment 154.

Preferably, the first provider practitioner computing system 108A will receive a notification when a patient 116 is assigned to the first provider practitioner pool 132A associated with the first provider practitioner computing system 108A. Notification details may be generated in the Firebase Firestore service. After a notification is generated, the user interface shows the notification and the first provider practitioner 112A can interact with the notification to view details or dismiss the notification. For example, the notification may include any note(s) from the provider staff member 110. Preferably, the application logic shows the notification only once and only for the first provider practitioner 112A.

In one embodiment, as described further below, a method of providing patient-centered medical information may be implemented for the patient 116 while the patient 116 is in the first provider practitioner pool 132A awaiting their consultation.

A provider practitioner 112A, 112B... 112N (e.g. a doctor) can select a patient 116 with whom the provider practitioner 112A, 112B ...112N wishes to initiate a consultation, for example from a list on a suitable user interface on the provider practitioner computing system 108A, 108B...108N. Thus, the telemedicine server system 102 can receive, from the first provider practitioner computing system 108A, a first consultation selection 156 of the patient 116 in the first provider practitioner pool 132A. Responsive to the first consultation selection 156, the telemedicine server system 102 initiates a first audiovisual consultation teleconference 158 between the patient computing system 114 and the first provider practitioner computing system 108A.

More particularly, when the first provider practitioner 112A indicates (by the first consultation selection 156) that s/he is ready to initiate the first audiovisual consultation teleconference 158 with the patient 116, the meeting token and the meeting access token is shared. These tokens are required to join the first audiovisual consultation teleconference 158. The frontend application includes functionality to check audiovisual connections to ensure that the audiovisual input/output methods used by the first provider practitioner computing system 108A are compatible. After confirming the device information, the first provider practitioner 112A and the patient 116 proceed with the first audiovisual consultation teleconference 158. In preferred embodiments, the provider practitioner 112A, 112B...112N can customize the web pages that will be presented to the patient 116 while the patient 116 awaits initiation of the first audiovisual consultation teleconference 158. For example, the provider practitioner 112A, 112B...112N can provide information about themselves, infographics, or other information.

During the first audiovisual consultation teleconference 158, the first provider practitioner 112A can consult with the patient 116 about symptoms, diagnosis and treatment. In some optional embodiments, peripheral devices may be used by the patient 116 to provide additional diagnostic information. Such peripheral devices may be coupled with the patient computing system 114 to communicate data to the first provider computing system 108, or the peripheral devices may be standalone devices and the patient may communicate the results orally or by holding up the peripheral device to a camera. Examples of suitable peripheral devices include, but are not limited to, blood pressure monitors/sensors, pulse oximeters, blood glucose monitors/sensors, and digital stethoscopes.

Optionally, the first provider practitioner 112A may cause the first provider practitioner computing system 108A invite one or more further participants to join the first audiovisual consultation teleconference 158, for example using a suitable user interface element. The additional participant(s) may be, for example, a second provider practitioner 112B, a practitioner from another provider outside the virtual clinic 104 (e.g. a specialist), or a family member of the patient 116. The invitation will generate a conference invite URL (different from the patient invite URL described above) which can connect the invitee’s web browser directly to the ongoing first audiovisual consultation teleconference 158. In one embodiment, the conference invite URL encodes information about the audiovisual consultation teleconference name, conference token, expiration of the conference invite URL, and other information. In one embodiment, the conference invite URL includes a JSON Web Token (JWT token) which contains the foregoing information. The computing system of the invitee may also undergo an audiovisual input/output compatibility check.

Also optionally, the first provider practitioner 112A may cause the first provider practitioner computing system 108A to pause or suspend the first audiovisual consultation teleconference 158 and initiate an intraprovider teleconference, for example between the first provider practitioner 112A and another provider practitioner 112B...112N or between the first provider practitioner 112A and a provider staff member 110.

Once the first provider practitioner 112A is satisfied with the first audiovisual consultation teleconference 158, the first provider practitioner 112A can end the first audiovisual consultation teleconference 158. The first provider practitioner 112A may, in association with ending the first audiovisual consultation teleconference 158, return the patient 116 to the provider waiting pool 130 (for example to speak with a provider staff member 110 to schedule another appointment or coordinate a prescription), or assign the patient 116 to a second provider practitioner 112B, or may simply end the first audiovisual consultation teleconference 158.

Where the first provider practitioner 112A wishes to assign the patient 116 to a second provider practitioner 112B, the first provider practitioner 112A may make a second practitioner selection assignment 160 assigning the patient 116 to the second provider practitioner 112B. For example, the first provider practitioner 112A may select the second provider practitioner 112B from a list of provider practitioners 112 on a user interface. The first provider practitioner 112A may include a note for the second provider practitioner 112B; this note would not be visible to the patient 116.

The telemedicine server system 102 receives, from the first provider practitioner computing system 108A, the second practitioner selection assignment 160 assigning the patient 116 to the second provider practitioner 112B. In response to the second practitioner selection assignment 160, the telemedicine server system 102 assigns the patient 116 to a second provider practitioner pool 132B associated with the second provider practitioner computing system 108B. As noted above, the second provider practitioner pool 132B exposes each patient 116 in the second provider practitioner pool 132B to the second provider practitioner computing system 108B. For example, a user interface on the frontend application executing on the second provider practitioner computing system 108B may display a list of patients 116 in the second provider practitioner pool 132B. A notification may be provided to the second provider practitioner 112B.

The telemedicine server system 102 will terminate the first audiovisual consultation teleconference 158 based on suitable input. In one embodiment, the telemedicine server system 102 will terminate the first audiovisual consultation teleconference 158 automatically in response to receiving the second practitioner selection assignment 160. In another embodiment, the telemedicine server system 102 will terminate the first audiovisual consultation teleconference 158 in response to a command from the first provider practitioner computing system 108A, either before or after the second practitioner selection assignment 160.

As noted above, a provider practitioner 112A, 112B... 112N can select a patient 116 with whom to initiate a consultation, for example from a list on a suitable user interface on the provider practitioner computing system 108A, 108B...108N. Accordingly, the telemedicine server system 102 can receive, from the second provider practitioner computing system 108B, a second consultation selection 162 of the patient 116 in the second provider practitioner pool 132B. Responsive to the second consultation selection 162, the telemedicine server system 102 initiates a second audiovisual consultation teleconference 164 between the patient computing system 114 and the second provider practitioner computing system 108B. As with the first audiovisual consultation teleconference 158 with the patient 116, the meeting token and the meeting access token is shared and the frontend application includes functionality to check audiovisual connections. Then, the second provider practitioner 112B and the patient 116 proceed with the second audiovisual consultation teleconference 164. Like the first provider practitioner 112A, the second provider practitioner 112B can consult with the patient 116 about symptoms, diagnosis and treatment, with optional use of peripheral devices. Also optionally, the second provider practitioner 112B may invite one or more further participants to join the second audiovisual consultation teleconference 164, or suspend the second audiovisual consultation teleconference 164 for an intraprovider teleconference. The second provider practitioner 112B may also assign the patient 116 to a third provider practitioner by making make a third practitioner selection assignment. The third provider practitioner may be different from the first provider practitioner 112A, but in some cases, the third provider practitioner may be the first provider practitioner 112A. For example, the first provider practitioner 112A may be a general practice small animal veterinarian who refers the patient 116 to a second provider practitioner 112B who is a veterinary ophthalmologist, who, after the second audiovisual consultation teleconference 164, returns the patient 116 to the first provider practitioner 112A. (Note here that in the veterinary context, the patient 116 may by the owner/custodian/guardian of a non-human animal who is the actual subject of medical care.) The second provider practitioner 112B may also simply end the second audiovisual consultation teleconference 164, or make a selection to return the patient to the admission pool 130.

In some embodiments, the first provider practitioner 112A may be a nurse and the second provider practitioner 112B may be a doctor. In such an embodiment, an intraprovider teleconference may be used to discuss a prescription or a treatment plan.

In some embodiments, the waiting pool 130 functions as an admission pool. In other embodiments, there may be one or more admissions pools which are separate and distinct from the waiting pool 130. The admissions pool(s) may be implemented as one or more admission queue(s). In these embodiments, responsive to the admission selection 150, the telemedicine server system 102 may assign the patient 116 to an admissions pool in association with initiating the audiovisual admissions teleconference 152 between the patient computing system 114 and the provider admitting computing system 106. Thus, in some optional embodiments using a queue-based implementation, the telemedicine server system 102 may maintain one or more specific admission queues associated with one or more provider staff members 110 and, upon receiving the admission selection 150 of the patient 116, the telemedicine server system 102 will move the patient 116 into the corresponding admission queue. After receiving the first practitioner selection assignment 154 from the provider admitting computing system 106, the telemedicine server system 102 would then move the patient 116 into the appropriate provider practitioner queue. These events would be updated in the Firebase Firestore system.

As described above, a number of notifications can be provided within the telemedicine server system 102. In one embodiment, notifications are given a unique identifier, with each notification record containing several elements of information to uniquely identify the notification such as notificationld, receiverld, timestamp, notificationType, among others. These attributes support the telemedicine server system 102 in sending notifications only to the intended user(s), and preventing multiple displays of the same notifications. Notifications may be, for example, by e-mail or SMS, or internal within the frontend application.

Internal messaging (e.g. among the provider admitting computing system(s) 106 and the provider practitioner computing system(s) 108) may also be supported by the telemedicine server system 102; these may optionally be encrypted. Attributes defined in the message record in the database uniquely identify every message sent; the content of the messages are discoverable in the Firebase database level and protected by Firebase platform security. Also optionally, the telemedicine server system 102 supports audiovisual teleconferences among the provider admitting computing system(s) 106 and the provider practitioner computing system(s) 108.

Optionally, where permitted by law, audiovisual admissions teleconferences and audiovisual consultation teleconferences may be recorded, with appropriate compliance procedures in place (e.g. explicit patient consent, HIPAA/PHIPA or other legal protections, etc.).

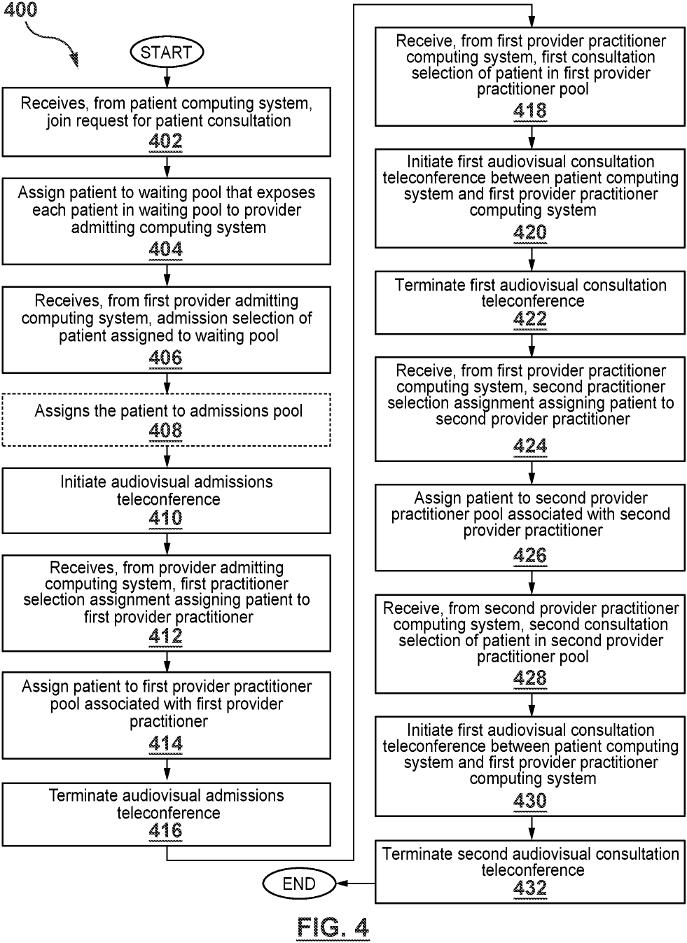

Reference is now made to FIG. 4, which is a flow chart showing an illustrative method 400 for administering telemedicine consultations according to the present disclosure. The method 400 may be implemented, for example, by the telemedicine server system 102 shown in FIG. 1.

At step 402, the method 400 receives, from a patient computing system, a join request for a patient consultation. The join request identifies a patient associated with the patient computing system, and may comprise a unique URL. At step 404, responsive to the join request, the method 400 assigns the patient to a waiting pool that exposes each patient in the waiting pool to a provider admitting computing system. At step 406, the method 400 receives, from the provider admitting computing system, an admission selection of the patient assigned to the waiting pool. At optional step 408, responsive to the admission selection, the method 400 assigns the patient to an admissions pool, and at step 410, also responsive to the admission selection, the method 400 initiates an audiovisual admissions teleconference between the patient computing system and the provider admitting computing system. Thus, where present, step 408 of assigning the patient to an admissions pool is carried out in association with step 410 of initiating the audiovisual admissions teleconference between the patient computing system and the provider admitting computing system. There may be a substantial time gap between step 404 and steps 406 to 410.

At step 412, the method 400 receives, from the provider admitting computing system, a first practitioner selection assignment assigning the patient to a first provider practitioner. At step 414, responsive to the first practitioner selection assignment, the method 400 assigns the patient to a first provider practitioner pool associated with a first provider practitioner computing system associated with a first provider practitioner. The first provider practitioner pool exposes each patient in the first provider practitioner pool to the first provider practitioner computing system.

At step 416 the method 400 terminates the audiovisual admissions teleconference, for example automatically responsive to the first practitioner selection assignment or in response to a manual command, and at step 418 the method 400 receives, from the first provider practitioner computing system, a first consultation selection of the patient in the first provider practitioner pool. Note that there may be a substantial gap in time between steps 416 and 418.

At step 420, responsive to the first consultation selection, the method 400 initiates a first audiovisual consultation teleconference between the patient computing system and the first provider practitioner computing system. At step 422 the method 400 terminates the first audiovisual consultation teleconference. Step 422 may be, for example, automatically responsive to a second practitioner selection assignment (step 424), in which case steps 422 and 424 may occur in reverse order, or substantially simultaneously, or step 422 may be in response to a manual command. In some embodiments, where the first provider practitioner does not wish to refer the patient to a second provider practitioner, the method may end after step 422. In other embodiments, where the first provider practitioner wishes to refer the patient to a second provider practitioner, the method may continue to steps 424 to 432.

At step 424, the method 400 receives, from the first provider practitioner computing system, a second practitioner selection assignment assigning the patient to a second provider practitioner. At step 426, responsive to the second practitioner selection assignment, the method 400 assigns the patient to a second provider practitioner pool associated with a second provider practitioner computing system. The second provider practitioner pool exposes each patient in the second provider practitioner pool to the second provider practitioner computing system.

At step 428 the method 400 receives, from the second provider practitioner computing system, a second consultation selection of the patient in the second provider practitioner pool. Note that there may be a substantial gap in time between steps 426 and 428.

At step 430, responsive to the second consultation selection, the method 400 initiates a second audiovisual consultation teleconference between the patient computing system and the second provider practitioner computing system, and at step 432, the method 400 terminates the second audiovisual consultation teleconference.

Importantly, audiovisual telemedicine is a computer-dependent field of technology, and the problems associated with conforming telemedicine to the workflow and experience that patients and medical practitioners have come to expect are by their nature a form of computer problem requiring the application of technology to generate a satisfactory resolution.

As can be seen from the above description, the systems and methods for telemedicine consultation described herein represent significantly more than merely using categories to organize, store and transmit information and organizing information through mathematical correlations. The systems and methods for telemedicine consultation are in fact an improvement to the technology of telemedicine, as they use a specific technical architecture in the form of distinct waiting pools and provider practitioner pools, to provide a natural replication of the familiar in-person clinical workflow, as shown in the chart below:

| | In-Person Clinical Workflow | Telemedicine Consultation (Present Disclosure) |
|---|---|---|
| 1 | Patient sees a receptionist to record basic info, date of birth, medical complaint, etc. | Patient enters a short URL |
| | | Patient has audiovisual admissions teleconference with provider staff member |
| 2 | Patient sees a nurse to record any symptoms, weight, blood pressure, etc. | Patient has first audiovisual consultation teleconference with first provider practitioner. |
| 3 | Patient sees the doctor. | Patient has second audiovisual consultation teleconference with second provider practitioner. |

The reference to “receptionist”, “nurse” and “doctor” in the chart above is merely illustrative and not limiting.

The virtual recreation of an in-clinic workflow in a telemedicine context facilitates the benefit of improved administrative efficiency by freeing highly trained provider practitioners from administrative tasks and allowing them to devote more time to patient consultation, improving the effectiveness and throughput of healthcare delivery. The systems and methods for telemedicine consultation described herein have physical existence and a discernible effect in that they result in multiple audiovisual teleconferences between geographically remote physical computing devices. Moreover, the systems and methods for telemedicine consultation are confined to telemedicine applications. Although confined to telemedicine applications, it is to be noted here that no particular method of medical or surgical treatment is described or claimed. Rather, the systems and methods for telemedicine consultation described herein support communication between patients and medical professionals.

As noted above, there may be a substantial time gap between certain steps, during which the patient 116 may be kept waiting. This is analogous to the context of a physical clinic where a patient may wait for some time in a reception area and/or an examination room. This provides an opportunity to present the patient with useful information.

In particular, because patient is already expecting to provide medical data in the context of telemedicine, it is possible to gather this medical data and use it precisely target the patient 116 during waiting periods with medical information that will be particularly useful to them, given their medical situation. For example, a Type 1 diabetic may be provided with information focused on the management of that condition, or a Celiac disease patient may be provided with information on foods to avoid.

There is a conundrum, however, in that precision targeting of information may invoke a “creep factor” in which the patient 116 is concerned about why particular information is being presented. The term “creep factor” is used to refer to consumer revulsion at a perception that their privacy is being invaded by the use of personal data to target (or even microtarget) them with advertising or other content. It can result in outright rejection of that content, undermining any potential benefit (e.g. making content more relevant to the consumer). This “creep factor” risk is particularly acute in a medical context, where the data at issue is of the utmost sensitivity. Yet, the ability to receive targeted medical information is potentially lifesaving. This is especially the case where an individual may be receptive to the information if it is made available in an impersonal context but unwilling to discuss or raise the matter with a physician due to embarrassment. Yet, if the patient 116 perceives an invasion of privacy by the presentation of such information, s/he may reject it due to this “creep factor”, with potentially life-threatening consequences.

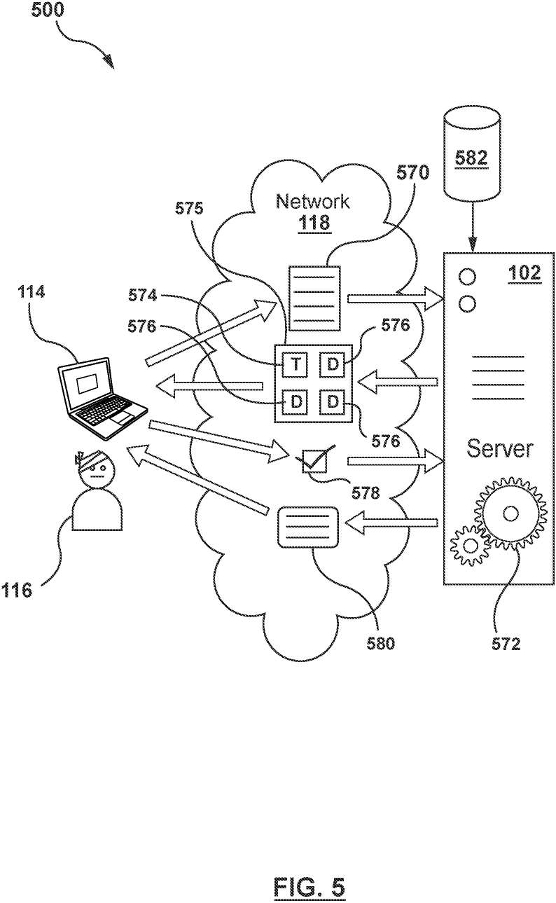

Reference is now made to FIG. 5, which illustrates a system 500 for providing patient-centered medical information. The system may be, or may comprise, the telemedicine server system 102, which communicates via network 118 with a patient computing system 114 associated with a patient 116. The telemedicine server system 102 receives medical data 570 from the patient computing system 114. The medical data 570 may include a date of birth, questions with free form answers (e.g. characters entered into a text box), or yes/no or multiple choice questions (e.g. pull-down menu selections, radio button or checkbox selections, etc.), among others, using the frontend application. The medical data 570 may be transmitted to a provider staff member 110 and/or to a provider practitioner 112A, 112B... 112N (see FIG. 1) for use during an audiovisual admissions teleconference 152 or an audiovisual consultation teleconference 158, 164. In a preferred embodiment, the medical data 570 is not drawn from any existing electronic medical record of the patient 116, but only from information directly input by the patient via the patient computing system 114. The telemedicine server system also communicates with the patient computing system 114 to provide any required privacy disclosures and obtain any required privacy consents for the use of the medical data 570.

The telemedicine server system 102, for example the backend application, executes program logic 572 to use the medical data 570 to select at least one targeted media item 574. There may be a single targeted media item 574 or a plurality of targeted media items 574. The targeted media item(s) 574 will be selected for relevance to the patient 116 based on the medical data 570. To select the targeted media item(s) 574, the program logic 572 may, for example, apply a decision tree to the medical data 570. In a decision tree embodiment, subsequent questions presented to the patient 116 may be determined by responses to previous questions. In another embodiment, the program logic 572 may apply a trained machine learning engine (e.g. a trained neural network) to the medical data 570, among other approaches. Natural language processing may be used for questions with free form answers.

The telemedicine server system 102 then presents to the patient 116, in a user interface of the patient computing system, an opportunity 575 to make a selection 578 of one of the targeted media item(s) 574 and at least one distractor media item 576. The distractor media item(s) 576 will be of lesser relevance to the patient, relative to the targeted media item(s) 574. The program logic 572 may use the medical data 570 to select the distractor media item(s) 576 to be of lesser relevance to the patient 116, relative to the targeted media item(s) 574. Thus, the distractor media item(s) 576 may have some relevance to the patient 116 based on the medical data 570, but less relevance than the targeted media item(s) 574. For example, if the medical data 570 indicates that the patient 116 has Type II diabetes, the targeted media item(s) 574 may include highly relevant information about suitable medications which are approved for the treatment of Type II diabetes, while the distractor media item(s) 576 may include more generalized information about healthy eating, weight loss, and exercise. Alternatively, the distractor media item(s) 576 may be selected randomly. For example, if the medical data 570 indicates that the patient 116 has Type II diabetes, while the targeted media item(s) 574 may include information about suitable medications, the distractor media item(s) 576 may include information of little or no relevance to Type II diabetes, such as information about the aging process and influenza vaccination. Preferably, the distractor media item(s) 576 are selected to avoid potentially sensitive medical topics, such as those relating to sexual health.

The telemedicine server system 102 receives the selection 578 from the patient computing system 114 and, responsive to the selection 578, presents, in the user interface of the patient computing system 114, the selected one 580 of the targeted media item(s) 574 and the distractor media item(s) 576.

The targeted media item(s) 574 and the distractor media item(s) 576 may be any type of media, including text, audio media, visual media, and audiovisual media, including without limitation web pages, podcasts, slideshows, motion comics, and movies, or any combination of the foregoing.

The targeted media item(s) 574 and the distractor media item(s) 576 may be stored in a storage element 582 coupled to the telemedicine server system 102 and communicated from the telemedicine server system 102 to the patient computing system 114. Although the media items 574, 576 can be stored locally, they do not need to be stored locally, and may be hosted by one or more third party media hosting services, such as (for example) YouTube or Rumble. The telemedicine server system 102 may simply maintain a directory of media items 574, 576 with suitable tags; the program logic 572 may use the medical data 570 to select the type(s) of tags for targeted media item(s) 574 and then use the tags to locate the relevant targeted media item(s) 574. In such an embodiment, the telemedicine server system 102 may cause the patient computing system 114 to access the media item(s) 574, 576 directly from one or more third party media hosting services.

Of note, the patient 116 is given an actual free choice among the targeted media item(s) 574 and the distractor media item(s) 576. Without being limited by theory, it is expected that in at least a significant minority of instances, the patient 116 will select the targeted media item(s), and thereby benefit from information that is relevant to the patient’s medical situation. Yet, the patient 116 will not perceive that any particular media item is being forced upon them, and the lesser relevance of the distractor media item(s) 576 will obviate a sense of targeting and thereby reduce the “creep factor”, while still permitting the relevant information to be presented. It is particularly important to note here that what is being described is in no way a method for managing human behavior; the patient 116 is at all times given a free choice among the targeted media item(s) 574 and the distractor media item(s) 576. Rather, what is described is a particular communication technology to mitigate the specific technological problem of “creep factor”. Because it arises in the context of the sophisticated data targeting enabled by computers, the risk of “creep factor” is necessarily a computer problem.

As can be seen from the above description, the patient-centric communication technology described herein represents significantly more than merely using categories to organize, store and transmit information and organizing information through mathematical correlations. The patient-centric communication technology is in fact an improvement to the technology of data-driven targeted communication, as they provide the ability to deliver such targeted communication with a reduced likelihood of rejection. Thus, the patient-centric communication technology described herein is a solution to the computer problem wherein “creep factor” may prevent a telemedicine patient from receiving medically relevant information. The patient-centric communication technology described herein has physical existence and a discernible effect in that it results in the communication and presentation of media items to a geographically remote physical computing device.

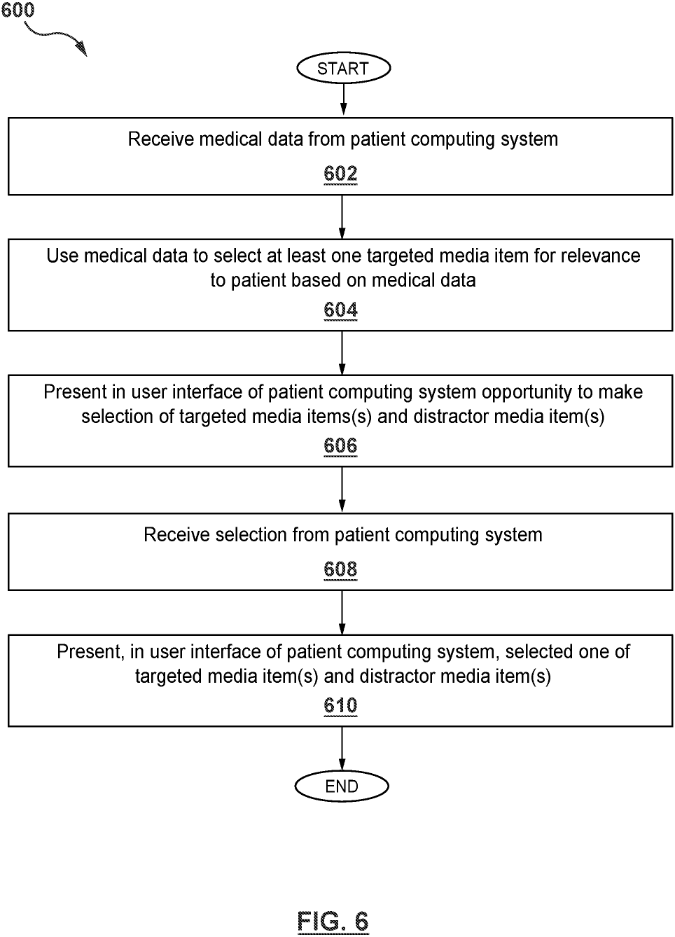

Referring now to FIG. 6, an illustrative method of providing patient-centered medical information while a patient waits to enter a virtual consultation in a telemedicine platform is shown generally at 600.

At step 602, the method 600 receives medical data from a patient computing system associated with the patient. At step 604, the method 600 uses the medical data to select at least one targeted media item for relevance to the patient based on the medical data. At step 606, the method 600 presents to the patient, in a user interface of the patient computing system, an opportunity to make a selection of one of the targeted media item(s) and at least one distractor media item of lesser relevance to the patient, relative to the targeted media item(s). At step 608, the method 600 receives the selection from the patient computing system. At step 610, responsive to the selection, the method 600 presents, in the user interface of the patient computing system, the selected one of the targeted media item(s) and the distractor media item(s).

The present technology may be embodied within a system, a method, a computer program product or any combination thereof. The computer program product may include a computer readable storage medium or media having computer readable program instructions thereon for causing a processor to carry out aspects of the present technology. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object oriented programming language or a conventional procedural programming language. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

Aspects of the present technology have been described above with reference to flowchart illustrations and/or block diagrams of methods, apparatus (systems) and computer program products according to various embodiments. In this regard, the flowchart and block diagrams in the Figures illustrate the architecture, functionality, and operation of possible implementations of systems, methods and computer program products according to various embodiments of the present technology. For instance, each block in the flowchart or block diagrams may represent a module, segment, or portion of instructions, which comprises one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Some specific examples of the foregoing may have been noted above but any such noted examples are not necessarily the only such examples. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, other programmable data processing apparatus, or other devices to function in a particular manner, such that the instructions stored in the computer readable storage medium produce an article of manufacture including instructions which implement aspects of the functions/acts specified in the flowchart and/or block diagram block or blocks. The computer readable program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

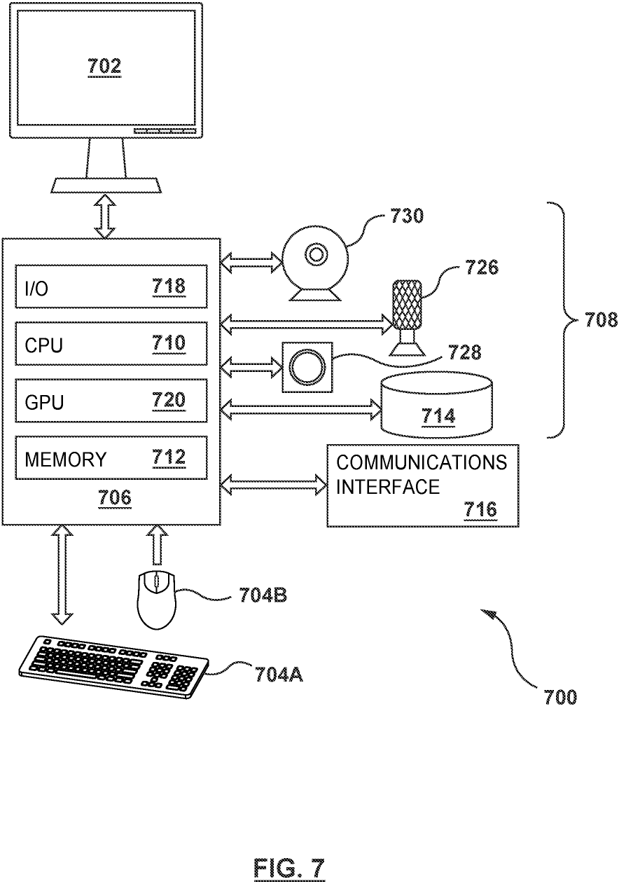

An illustrative computer system in respect of which the technology herein described may be implemented is presented as a block diagram in FIG. 7. The illustrative computer system is denoted generally by reference numeral 700 and includes a display 702, input devices in the form of keyboard 704A and pointing device 704B, computer 706 and external devices 708. While pointing device 704B is depicted as a mouse, it will be appreciated that other types of pointing device, or a touch screen, may also be used.

The computer 706 may contain one or more processors or microprocessors, such as a central processing unit (CPU) 710. The CPU 710 performs arithmetic calculations and control functions to execute software stored in an internal memory 712, preferably random access memory (RAM) and/or read only memory (ROM), and possibly additional memory 714. The additional memory 714 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 714 may be physically internal to the computer 706, or external as shown in FIG. 7, or both.

The computer system 700 may also include other similar means for allowing computer programs or other instructions to be loaded. Such means can include, for example, a communications interface 716 which allows software and data to be transferred between the computer system 700 and external systems and networks. Examples of communications interface 716 can include a modem, a network interface such as an Ethernet card, a wireless communication interface, or a serial or parallel communications port. Software and data transferred via communications interface 716 are in the form of signals which can be electronic, acoustic, electromagnetic, optical or other signals capable of being received by communications interface 716. Multiple interfaces, of course, can be provided on a single computer system 700.

Input and output to and from the computer 706 is administered by the input/output (I/O) interface 718. This I/O interface 718 administers control of the display 702, keyboard 704A, external devices 708 and other such components of the computer system 700. The computer 706 also includes a graphical processing unit (GPU) 720. The latter may also be used for computational purposes as an adjunct to, or instead of, the (CPU) 710, for mathematical calculations.

The external devices 708 include a microphone 726, a speaker 728 and a camera 730. Although shown as external devices, they may alternatively be built in as part of the hardware of the computer system 700, and may be used, for example, in the audiovisual admissions teleconference 152 or audiovisual consultation teleconferences 158, 164.

The various components of the computer system 700 are coupled to one another either directly or by coupling to suitable buses.

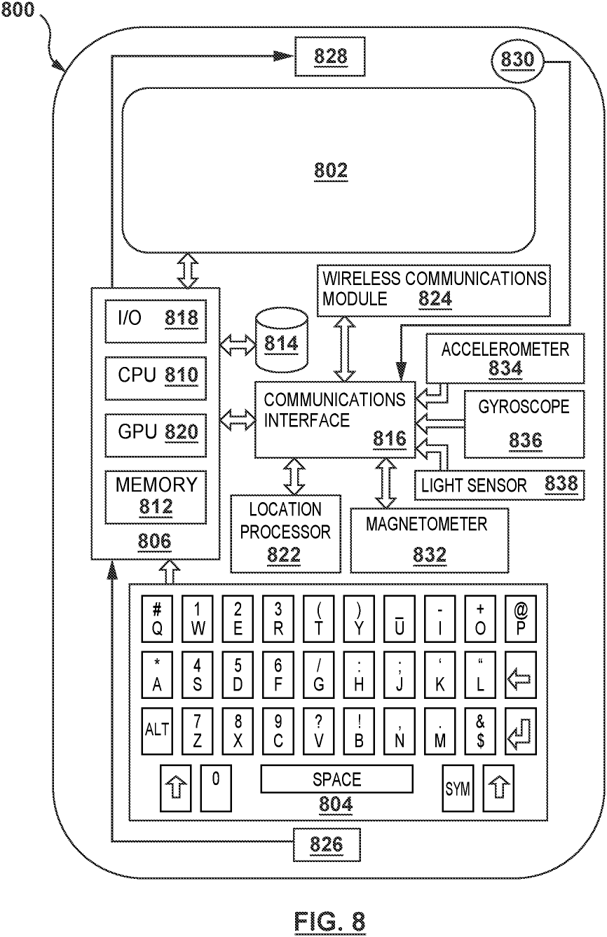

FIG. 8 shows an illustrative networked mobile wireless telecommunication computing device in the form of a smartphone 800. The smartphone 800 includes a display 802, an input device in the form of keyboard 804 and an onboard computer system 806. The display 802 may be a touchscreen display and thereby serve as an additional input device, or as an alternative to the keyboard 804. The onboard computer system 806 comprises a central processing unit (CPU) 810 having one or more processors or microprocessors for performing arithmetic calculations and control functions to execute software stored in an internal memory 812, preferably random access memory (RAM) and/or read only memory (ROM) is coupled to additional memory 814 which will typically comprise flash memory, which may be integrated into the smartphone 800 or may comprise a removable flash card, or both. The smartphone 800 also includes a communications interface 816 which allows software and data to be transferred between the smartphone 800 and external systems and networks. The communications interface 816 is coupled to one or more wireless communication modules 824, which will typically comprise a wireless radio for connecting to one or more of a cellular network, a wireless digital network or a Wi-Fi network. The communications interface 816 will also typically enable a wired connection of the smartphone 800 to an external computer system. A microphone 826 and speaker 828 are coupled to the onboard computer system 806 to support the telephone functions managed by the onboard computer system 806, and a location processor 822 (e.g. including GPS receiver hardware) may also be coupled to the communications interface 816 to support navigation operations by the onboard computer system 806. One or more cameras 830 (e.g. front-facing and/or rear facing cameras) may also be coupled to the onboard computer system 806, as may be one or more of a magnetometer 832, accelerometer 834, gyroscope 836 and light sensor 838. Input and output to and from the onboard computer system 806 is administered by the input/output (I/O) interface 818, which administers control of the display 802, keyboard 804, microphone 826, speaker 828, camera 830, magnetometer 832, accelerometer 834, gyroscope 836 and light sensor 838. The onboard computer system 806 may also include a separate graphical processing unit (GPU) 820. The various components are coupled to one another either directly or by coupling to suitable buses.

The microphone 826, speaker 828 and camera(s) 830 may be used, for example, in the audiovisual admissions teleconference 152 or audiovisual consultation teleconferences 158, 164.

The term “computer system”, “data processing system” and related terms, as used herein, is not limited to any particular type of computer system and encompasses servers, desktop computers, laptop computers, networked mobile wireless telecommunication computing devices such as smartphones, tablet computers, as well as other types of computer systems.

Thus, computer readable program code for implementing aspects of the technology described herein may be contained or stored in the memory 812 of the onboard computer system 806 of the smartphone 800 or the memory 712 of the computer 706, or on a computer usable or computer readable medium external to the onboard computer system 806 of the smartphone 800 or the computer 706, or on any combination thereof.

Finally, the terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

One or more currently preferred embodiments have been described by way of example. It will be apparent to persons skilled in the art that a number of variations and modifications can be made without departing from the scope of the claims. In construing the claims, it is to be understood that the use of a computer to implement the embodiments described herein is essential.

What is claimed is:

1. A method for administering telemedicine consultations, comprising:
receiving, from a patient computing system, a join request for a patient consultation, wherein the join request identifies a patient associated with the patient computing system;
responsive to the join request, assigning the patient to a waiting pool, wherein the waiting pool exposes each patient in the waiting pool to a provider admitting computing system; and
receiving, from the provider admitting computing system, an admission selection of the patient assigned to the waiting pool;
responsive to the admission selection, initiating an audiovisual admissions teleconference between the patient computing system and the provider admitting computing system;
receiving, from the provider admitting computing system, a first practitioner selection assignment assigning the patient to a first provider practitioner;
responsive to the first practitioner selection assignment, assigning the patient to a first provider practitioner pool associated with a first provider practitioner computing system associated with a first provider practitioner, wherein the first provider practitioner pool exposes each patient in the first provider practitioner pool to the first provider practitioner computing system;
terminating the audiovisual admissions teleconference;
receiving, from the first provider practitioner computing system, a first consultation selection of the patient in the first provider practitioner pool; and
responsive to the first consultation selection, initiating a first audiovisual consultation teleconference between the patient computing system and the first provider practitioner computing system.

2. The method of claim 1, wherein:
assigning the patient to a waiting pool comprises assigning a patient object representing the patient to a waiting queue; and
assigning the patient to the first provider practitioner pool comprises assigning the patient object representing the patient to a first provider practitioner queue;
wherein the waiting queue and the first provider practitioner queue are distinct from one another.

3. The method of claim 1, wherein:
assigning the patient to a waiting pool comprises assigning a patient object representing the patient to a waiting queue; and
assigning the patient to a first provider practitioner pool comprises assigning the patient object representing the patient to a common practitioner provider queue and assigning a first provider practitioner tag to the patient object, wherein the first provider practitioner tag associates the patient object with the first provider practitioner computer system;
wherein the waiting queue and the common provider practitioner queue are distinct from one another.

4. The method of claim 1, wherein:
assigning the patient to a waiting pool comprises assigning a patient object representing the patient to a single common queue and assigning an admitting tag to the patient object; and
assigning the patient to the first provider practitioner pool comprises maintaining the patient object in the single common queue and assigning a first provider practitioner tag to the patient object, wherein the first provider practitioner tag associates the patient object with the first provider practitioner computer system.

5. The method of claim 1, further comprising:
receiving, from the first provider practitioner computing system, a second practitioner selection assignment assigning the patient to a second provider practitioner;
responsive to the second practitioner selection assignment, assigning the patient to a second provider practitioner pool associated with a second provider practitioner computing system, wherein the second provider practitioner pool exposes each patient in the second provider practitioner pool to the second provider practitioner computing system;
terminating the first audiovisual consultation teleconference;
receiving, from the second provider practitioner computing system, a second consultation selection of the patient in the second provider practitioner pool; and
responsive to the second consultation selection, initiating a second audiovisual consultation teleconference between the patient computing system and the second provider practitioner computing system.

6. The method of claim 5, wherein:
the first provider practitioner pool exposes each patient in the first provider practitioner pool to the second provider practitioner computing system; and
the second provider practitioner pool exposes each patient in the second provider practitioner pool to the first provider practitioner computing system.

7. The method of claim 1, wherein the waiting pool further exposes each patient in the waiting pool to the first provider practitioner computing system.

8. The method of claim 1, further comprising, responsive to the admission selection, assigning the patient to an admissions pool in association with initiating the audiovisual admissions teleconference between the patient computing system and the provider admitting computing system.

9. A telemedicine computing system, comprising:
a telemedicine server system, the server system communicatively coupled to:
a patient computing system;
a provider admitting computing system; and
a first provider practitioner computing system associated with a first provider practitioner;
the telemedicine server system adapted to:
maintain a provider waiting pool and expose each patient in the provider waiting pool to at least one of the at least one provider admitting computing system; and
maintain at least one provider practitioner pool and expose each patient in the provider practitioner pool to at least one of the at least one provider practitioner computing system;
the telemedicine server system further adapted to:
receive, from one of the at least one patient computing system, a join request for a patient consultation, wherein the join request identifies a patient associated with the patient computing system;
responsive to the join request, assign the patient to the provider waiting pool;
receive, from one of the at least one provider admitting computing system, an admission selection of the patient assigned to the provider waiting pool;
responsive to the admission selection, initiate an audiovisual admissions teleconference between the one of the at least one patient computing system and the one of the at least one provider admitting computing system;
receive, from the provider admitting computing system, a first practitioner selection assignment assigning the patient to the first provider practitioner;
responsive to the first practitioner selection assignment, assign the patient to a first provider practitioner pool associated with the first provider practitioner computing system, wherein the first provider practitioner pool exposes each patient in the first provider practitioner pool to the first provider practitioner computing system;
terminate the audiovisual admissions teleconference;
receive, from the first provider practitioner computing system, a first consultation selection of the patient in the first provider practitioner pool; and
responsive to the first consultation selection, initiate a first audiovisual consultation teleconference between the patient computing system and the first provider practitioner computing system.

10. The telemedicine computing system of claim 9, wherein the telemedicine computing system is adapted to:
assign the patient to a waiting pool by assigning a patient object representing the patient to a waiting queue; and
assign the patient to the first provider practitioner pool by assigning the patient object representing the patient to a first provider practitioner queue;
wherein the waiting queue and the first provider practitioner queue are distinct from one another.

11. The telemedicine computing system of claim 9, wherein the telemedicine computing system is adapted to:
assign the patient to a waiting pool by assigning a patient object representing the patient to a waiting queue; and
assign the patient to a first provider practitioner pool by assigning the patient object representing the patient to a common practitioner provider queue and assigning a first provider practitioner tag to the patient object, wherein the first provider practitioner tag associates the patient object with the first provider practitioner computer system;
wherein the waiting queue and the common provider practitioner queue are distinct from one another.

12. The telemedicine computing system of claim 9, wherein the telemedicine computing system is adapted to:
assign the patient to a waiting pool by assigning a patient object representing the patient to a single common queue and assigning an admitting tag to the patient object; and
assign the patient to the first provider practitioner pool by maintaining the patient object in the single common queue and assigning a first provider practitioner tag to the patient object, wherein the first provider practitioner tag associates the patient object with the first provider practitioner computer system.

13. The telemedicine computing system of claim 9, wherein the telemedicine computing system is further adapted to:
receive, from the first provider practitioner computing system, a second practitioner selection assignment assigning the patient to a second provider practitioner;
responsive to the second practitioner selection assignment, assign the patient to a second provider practitioner pool associated with a second provider practitioner computing system, wherein the second provider practitioner pool exposes each patient in the second provider practitioner pool to the second provider practitioner computing system;
terminate the first audiovisual consultation teleconference;
receive, from the second provider practitioner computing system, a second consultation selection of the patient in the second provider practitioner pool; and
responsive to the second consultation selection, initiate a second audiovisual consultation teleconference between the patient computing system and the second provider practitioner computing system.

14. The telemedicine computing system of claim 13, wherein:
the first provider practitioner pool exposes each patient in the first provider practitioner pool to the second provider practitioner computing system; and
the second provider practitioner pool exposes each patient in the second provider practitioner pool to the first provider practitioner computing system.

15. The telemedicine computing system of claim 9, wherein the waiting pool further exposes each patient in the waiting pool to the first provider practitioner computing system.

16. The telemedicine computing system of claim 9, wherein the telemedicine computing system is further adapted to, responsive to the admission selection, assign the patient to an admissions pool in association with initiating the audiovisual admissions teleconference between the patient computing system and the provider admitting computing system.

17. A method of providing patient-centered medical information, comprising, while a patient waits to enter a virtual consultation in a telemedicine platform:
receiving medical data from a patient computing system associated with the patient;
using the medical data to select at least one targeted media item, wherein the at least one targeted media item is selected for relevance to the patient based on the medical data;
presenting to the patient, in a user interface of the patient computing system, an opportunity to make a selection of one of:
the at least one targeted media item; and
at least one distractor media item of lesser relevance to the patient, relative to the at least one targeted media item;
receiving the selection from the patient computing system; and
responsive to the selection, presenting, in the user interface of the patient computing system, the selected one of:
the at least one targeted media item; and
the at least one distractor media item.

18. The method of claim 17, further comprising using the medical data to select the at least one distractor media item to be of lesser relevance to the patient, relative to the at least one targeted media item.

19. The method of claim 17, wherein the at least one distractor media item is selected randomly.

20. The method of claim 17, wherein using the medical data to select at least one targeted media item comprises applying a decision tree to the medical data.

21. The method of claim 17, wherein using the medical data to select at least one targeted media item comprises applying a trained machine learning engine to the medical data.

22. A telemedicine computing system adapted for providing patient-centered medical information while a patient waits to enter a virtual consultation in a telemedicine platform, the telemedicine computing system comprising:
a telemedicine server system communicatively coupled to a patient computing system;
the telemedicine server system adapted to:
receive medical data from a patient computing system associated with the patient;
use the medical data to select at least one targeted media item, wherein the at least one targeted media item is selected for relevance to the patient based on the medical data;
present to the patient, in a user interface of the patient computing system, an opportunity to make a selection of one of:
the at least one targeted media item; and
at least one distractor media item of lesser relevance to the patient, relative to the at least one targeted media item;
receive the selection from the patient computing system; and
responsive to the selection, present, in the user interface of the patient computing system, the selected one of:
the at least one targeted media item; and
the at least one distractor media item.

23. The telemedicine computing system of claim 22, wherein the telemedicine computing system is adapted to use the medical data to select the at least one distractor media item to be of lesser relevance to the patient, relative to the at least one targeted media item.

24. The telemedicine computing system of claim 22, wherein the telemedicine computing system is adapted to select the at least one distractor media item randomly.

25. The telemedicine computing system of claim 22, wherein the telemedicine computing system is adapted to use the medical data to select at least one targeted media item by applying a decision tree to the medical data.

26. The telemedicine computing system of claim 22 telemedicine computing system, wherein the telemedicine computing system is adapted to use the medical data to select at least one targeted media item by applying a trained machine learning engine to the medical data.
